(19) United States
(12) Patent Application Publication
(10) Pub. No.: US 2008/0140099 A1
(43) Pub. Date: Jun. 12, 2008

(54) PYLORIC PLUG

(75) Inventors: Ragae Ghabrial, Cincinnati, OH (US); Sean P. Conlon, Loveland, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ETHICON ENDO-SURGERY, INC., Cincinnati, OH (US)

(21) Appl. No.: 11/609,401

(22) Filed: Dec. 12, 2006

(51) Int. Cl. A61B 17/08

(52) U.S. Cl. 606/157; 600/116

(57) ABSTRACT

Devices and methods for transgastric surgery are provided. In one aspect, a surgical device is provided that includes an elongate member having a proximal end, a distal end, and an inner lumen extending through at least a portion thereof that is adapted to receive a fluid flow therethrough. A selectively expandable member is coupled to the distal end of the elongate member and is adapted to receive fluid from the elongate member. A mesh is disposed around at least a portion of the expandable member. The mesh has a pre-shaped configuration, and at least a portion of the mesh is adapted to be operatively positioned to occlude a portion of a hollow organ.

### FIELD OF THE INVENTION

The present invention relates to surgical devices, and in particular to devices and methods for transgastric surgery.

### BACKGROUND OF THE INVENTION

Transgastric surgery is one type of minimally invasive surgery in which a surgeon can access the stomach via natural openings (e.g., mouth, anus) of the body and through the peritoneal lining of the abdominal cavity. In particular, once within the stomach, the surgeon can form an opening through the stomach wall to access the abdominal cavity. The surgeon can then use this opening to perform a variety of surgical procedures on organs located in the abdominal cavity.

A variety of techniques can be used to form an opening in the stomach wall. Typically, the patient's stomach is insufflated prior to accessing and forming the opening through the stomach wall. This causes the vital organs within the peritoneal cavity to move away from the stomach wall. As a result, the surgeon can puncture the wall of the stomach without the risk of injury to the organs within the peritoneal cavity. In some instances, where more time is needed to puncture the stomach wall, it will be necessary to inflate the stomach for a longer period of time. The longer the stomach is inflated, the greater the risk that fluid and air will escape through the pyloric valve and into the intestines. This can cause the intestines to inflate and fill the peritoneal cavity, thus hindering visualization and operability during the procedure.

Accordingly, there remains a need for improved devices and methods for transgastric surgery.

### SUMMARY OF THE INVENTION

The present invention provides devices and methods for transgastric surgery. In one aspect, a surgical device includes an elongate member having a proximal end, a distal end, and an inner lumen extending through at least a portion thereof that is adapted to receive a fluid flow therethrough. A selectively expandable member is coupled to the distal end of the elongate member and is adapted to receive fluid from the elongate member. A mesh is disposed around at least a portion of the expandable member. The mesh has a pre-shaped configuration, and at least a portion of the mesh is adapted to be operatively positioned to occlude a portion of a hollow organ. In one embodiment, the expandable member is fully contained within the mesh such that the expandable member conforms to the pre-shaped configuration of the mesh when the expandable member is expanded.

The expandable member can have a variety of configurations, and in one embodiment it is annularly shaped. In other embodiments, it can be a balloon-like. The expandable member can also have a central opening formed therein, and a slit can be formed within a wall of the central opening that is configured to deliver fluid to the expandable member.

A variety of techniques can be used to deliver fluid to the expandable member, and in one embodiment, the elongate member can be disposed within at least a portion of the central opening of the expandable member. The elongate member can have an inner lumen with an outlet, and this outlet can be aligned with the slit formed in the central opening of such that fluid can flow from the elongate member to the expandable member. In one embodiment, a valve can be disposed between the outlet and an inlet of the expandable member to maintain the fluid within the expandable member. While the valve can have a variety of configurations, the valve can be a one-way valve.

The expandable member can also be frangibly coupled to the distal end of the elongate member such that it can break away from the elongate member and remain in the tissue. In one embodiment, the elongate member includes a break point that is proximal to the expandable member that enables the expandable member to be frangibly coupled to the elongate member.

In another aspect, a kit for transgastric surgery is provided that includes an insertion member having at least one delivery channel extending therethrough. The insertion member is configured to be delivered translumenally to a surgical site within a patient. The kit also includes an elongate member configured to be removably disposed within the delivery channel. The elongate member has proximal and distal ends and a fluid-conveying lumen that extends through at least a portion thereof. Additionally, an anchoring member is coupled to the distal end of the elongate member and includes an expandable member contained within a mesh. In one embodiment, the expandable member can be fully contained within the mesh such that the expandable member conforms to a pre-shaped configuration of the mesh when the expandable member is expanded.

The anchoring member can have a variety of configurations. In one embodiment, at least a portion of the member has a configuration such that it is adapted to occlude a portion of a hollow organ when it is in an expanded position. By way of non-limiting example, the mesh can have an enlarged proximal portion and a narrowed distal portion. The insertion member can also have a variety of configurations, however in one embodiment, it can be an endoscope.

In another aspect, a method for transgastric surgery is provided that includes positioning a portion of an anchor assembly located on a distal end of an elongate member within an opening of a pyloric valve of a patient's stomach. The method also includes expanding an expandable member of the anchor assembly such that a mesh disposed around the expandable member engages and anchors the expandable member within the opening of the pyloric valve. This can temporarily block fluid flow from the stomach through the pyloric valve. The method can also include, prior to positioning, inserting the elongate member through an insertion member extending through a body lumen to position the anchor assembly within the stomach.

In one embodiment, the method can include partially inflating the expandable member to a diameter that is substantially equal to a diameter of the pyloric valve, pushing a portion of the expandable member into the pyloric valve, and inflating the expandable member such that the mesh disposed around the expandable member engages and anchors the expandable member within the pyloric valve to block fluid flow from the stomach to the pyloric valve.

### BRIEF DESCRIPTION OF THE DRAWINGS

The invention will be more fully understood from the following detailed description taken in conjunction with the accompanying drawings, in which:

FIG. 1A is a perspective view of one embodiment of a device for transgastic surgery having an anchoring assembly in a deflated position;

FIG. 1B is a perspective view of the device of FIG. 1A with the anchoring assembly in an expanded position;

FIG. 1C is an exploded view of a distal end of the device of FIG. 1B;

FIG. 2A is a cross-sectional view of one embodiment of a distal end of the elongate member of the device of FIG. 1C taken across line A-A;

FIG. 2B is a perspective view of one embodiment of a break point that is formed on the elongate member of the device of FIG. 2A;

FIG. 3 is a perspective view of one embodiment of a valve for sealing fluid within an anchoring assembly of the device of FIG. 1C;

FIG. 4 is a cross-sectional view of the expandable member of the device of FIG. 1C taken across line C-C;

FIG. 5 is a cross-sectional view of the distal end of the device of FIG. 1B taken across line B-B;

FIG. 6A is a schematic illustrating a side view of the device of FIGS. 1A-1C, showing the anchoring assembly in an insertion position;

FIG. 6B is a schematic illustrating the device of FIG. 6A upon insertion into a stomach and upon inflation of the anchoring assembly to engage a pyloric valve; and

FIG. 6C is a schematic illustrating side view of the device of FIG. 6A, showing the anchoring assembly separated from the elongate member, such that it remains disposed in the pyloric valve.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides devices and methods for occluding fluid (i.e., liquid or gas) flow from a hollow organ. In one embodiment, the device includes an elongate member having an anchoring assembly formed on a distal end thereof. The anchoring assembly can include an expandable member that is surrounded by a pre-formed mesh. In use, the device can be positioned within a hollow organ and the expandable member can be inflated such that it expands to the configuration of the pre-formed mesh and blocks an opening of the hollow organ. The anchoring assembly can optionally be severed from the elongate member, such that it can remain in place throughout the surgical procedure. Following completion of the surgical procedure, the expandable member can be deflated and the anchoring assembly removed from the organ. While the exemplary embodiment illustrates the device being used in the context of trans-gastric surgeries, such as for occluding the pyloric valve during insufflation of the stomach, one skilled in the art will appreciate that the device can be used with a variety of surgical procedures to occlude a variety of valves and hollow organs within a human body. One skilled in the art will also appreciate that the present invention has applications in conventional endoscopic and open surgical instrumentation as well applications in robotic-assisted surgery.

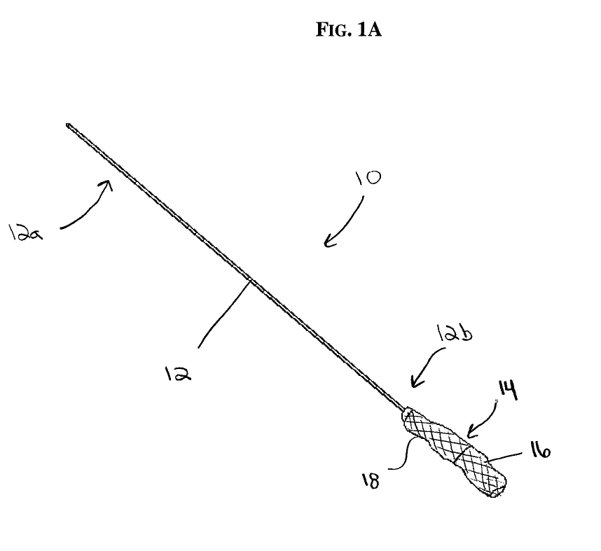

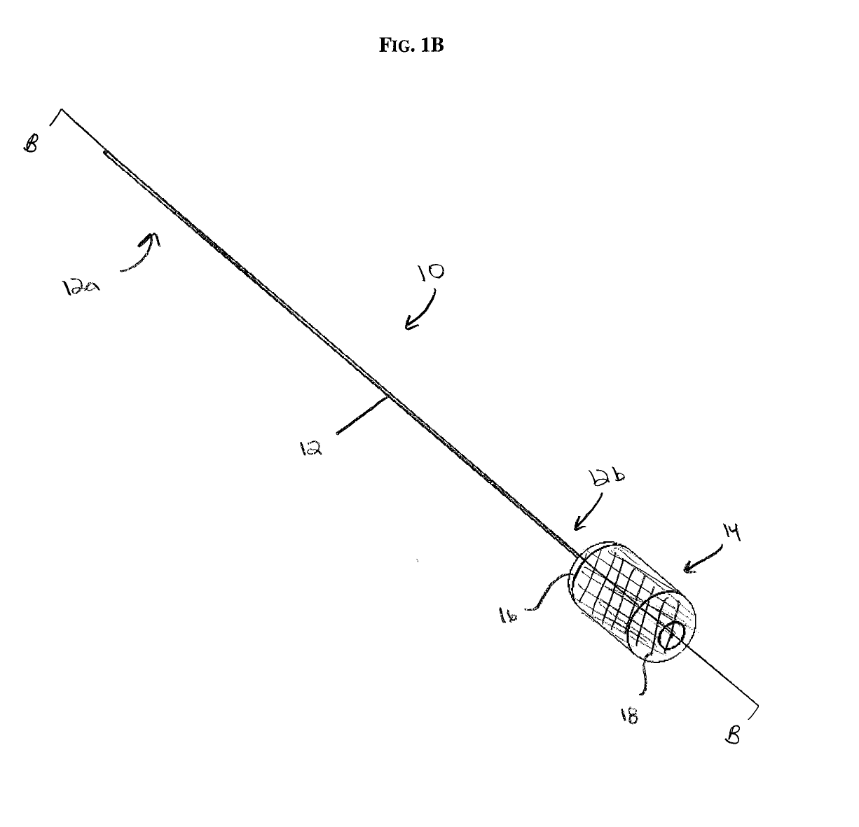

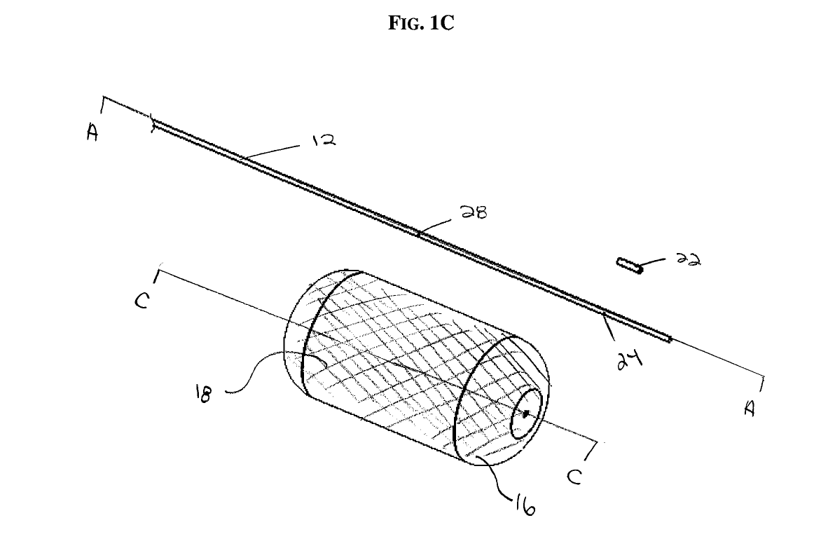

FIGS. 1A-1B illustrate one embodiment of a surgical device 10 that includes an elongate member 12 having proximal and distal ends 12a, 12b. An anchoring assembly 14 is formed on the distal end 12b of the elongate member 12, and it includes an expandable member 16 that is surrounded by a mesh 18. FIG. 1A illustrates the device 10 in the delivery configuration, in which the anchoring assembly 14 is in the deflated position. As shown, the device 10 is substantially linear to facilitate ease of delivery to the target site in the tissue. Following the placement of the device 10 within tissue, the expandable member 16 of the anchoring assembly 14 can be inflated such that the anchoring assembly 14 moves from the deflated position to an expanded position. As shown in FIG. 1B, the expandable member 16 expands to the shape of the mesh 18, and can be used to occlude an opening of a hollow organ, as will be discussed in more detail below.

The elongate member 12 can have virtually any configuration that allows it to be laparoscopically or endoscopically inserted to a surgical site. The member 12 can be substantially cylindrical and sufficiently strong, yet flexible enough to bend during insertion through a tortuous lumen. In one embodiment, the elongate member 12 is preferably substantially solid to provide structural support, but can include a lumen 20 formed therein and extending therethrough, as will be discussed in more detail below. In one embodiment, the proximal end 12a of the elongate member 12 can be adapted to couple to a fluid delivery device, as is known in the art. Alternatively or additionally, the member 12 can include markings (not shown) formed thereon to facilitate positioning the device 10 within a hollow organ. While the member 12 can have a variety of sizes, the size of the member 12 generally depends upon the desired application of the device 10. In an exemplary embodiment, where the device 10 is used during trans-gastric surgeries, the member 12 can have a diameter in the range of about 2.8 mm to 4.0 mm.

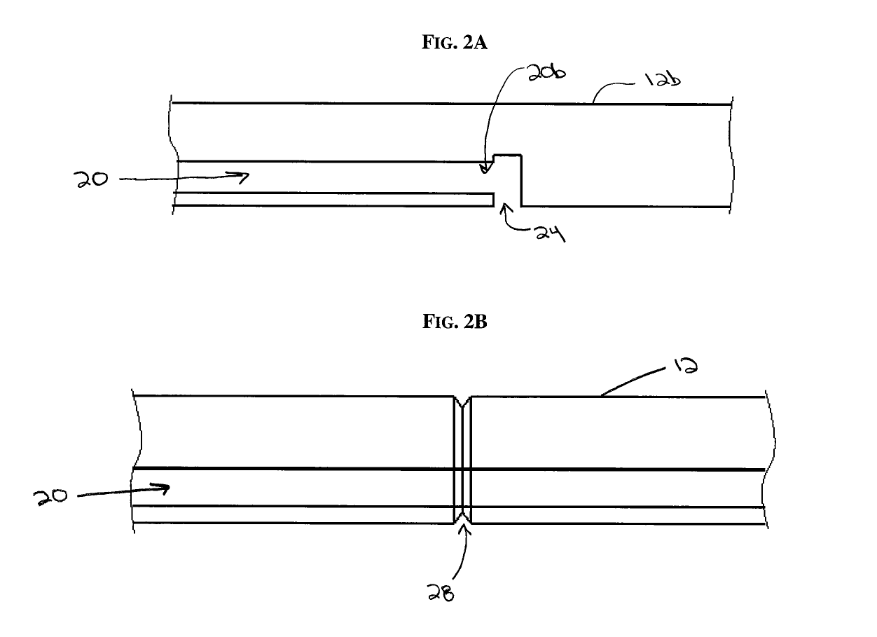

As noted above, a lumen 20 can extend through the elongate member 12. The lumen 20 can have a variety of shapes and sizes, but in an exemplary embodiment it has a diameter that is significantly smaller than a diameter of the elongate member 12 so as not to interfere with the structural integrity thereof. The lumen 20 can also extend fully or only partially between the proximal and distal ends 12a, 12b of the elongate member 12 so that a fluid can be delivered to the expandable member 16, as will be discussed in more detail below. FIG. 2A illustrates one exemplary embodiment of the fluid-delivering lumen 20 that includes a distal end 20b that extends through a sidewall of the elongate member 12 to form an outlet or an opening 24. In use, and as will be discussed in more detail below, fluid can pass through the lumen 20, out of the opening 24, and through an inlet or a slit 36 of the expandable member 16 to effect expansion of the expandable member 16.

The elongate member 12 can also optionally be adapted to separate from the anchoring assembly 14 upon the slight application of force. While a variety of techniques can be used to effect separation of the device 10, in one embodiment, the anchoring assembly 14 is frangibly coupled to the elongate member 12. For example, as shown in FIG. 2B, the elongate member 12 can include a break point 28 that is formed thereon and located proximal to the anchoring assembly 14. A variety of techniques can be used to form the break point. In one embodiment, the break point 28 can be formed from an area of weakened or thinned material. Alternatively, the break point can be formed by a score(s) or perforation(s) that is formed in at least one section of the elongate member. One skilled in the art will appreciate the variety of techniques that can be used to form the break point. In use, force can be applied to cause the anchor assembly 14 to sever from the elongate member 12 at the break point 28. This allows the elongate member 12 to be removed from a delivery channel of an insertion member while the anchoring assembly 14 remains within the hollow organ during a surgical procedure.

As noted above, an anchoring assembly 14 is formed on the distal end 12b of the elongate member 12. The anchoring member 14 includes an expandable member 16 having a mesh 18 that is disposed around at least a portion thereof. FIGS. 1C and 3-4 illustrate the expandable member 16 in more detail. While the expandable member 16 can have a variety of configurations, it is generally adapted to selectively expand upon the receipt of fluid. As shown, the expandable member 16 is annularly shaped, however it can have other shapes, such as an oblong shape, to allow the expandable member 16 to match the opening in an organ when it is in the expanded position. One skilled in the art will appreciate that the expandable member 16 can be formed from any material that is biocompatible and able to be expanded. Exemplary materials include silicone or polyurethane.

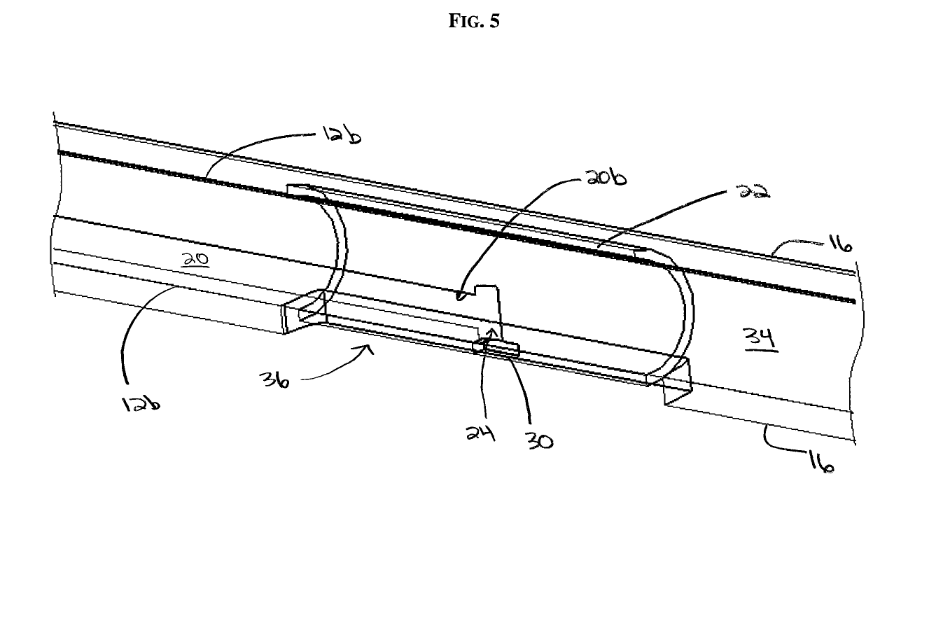

The expandable member 16 can have a variety of features that facilitate the selective expansion thereof. In one embodiment and as shown in FIG. 4, the expandable member 16 is annular and includes a central opening 34 that is formed therein and extends through at least a portion thereof. The central opening 34 is defined by an inner tubular wall that extends through the expandable member 16, and a valve 22 is adapted to be positioned adjacent to the inner tubular wall, as will be discussed in more detail below. The central opening 34 can have any shape and size, but should be adapted to receive at least a portion of the elongate member 12. An inlet or a slit 36 can be formed in a wall 35 of the central opening 34. While the slit 36 can have a variety of configurations, in an exemplary embodiment the slit 36 is substantially oblong, and has a size that is equal to or greater than a size of the opening 24 in the lumen 20 of the elongate member 12. In use, the slit 36 is adapted to receive fluid from the elongate member 12 and to deliver fluid to the expandable member 16 so that it can fill the expandable member 16, as will be discussed in more detail below.

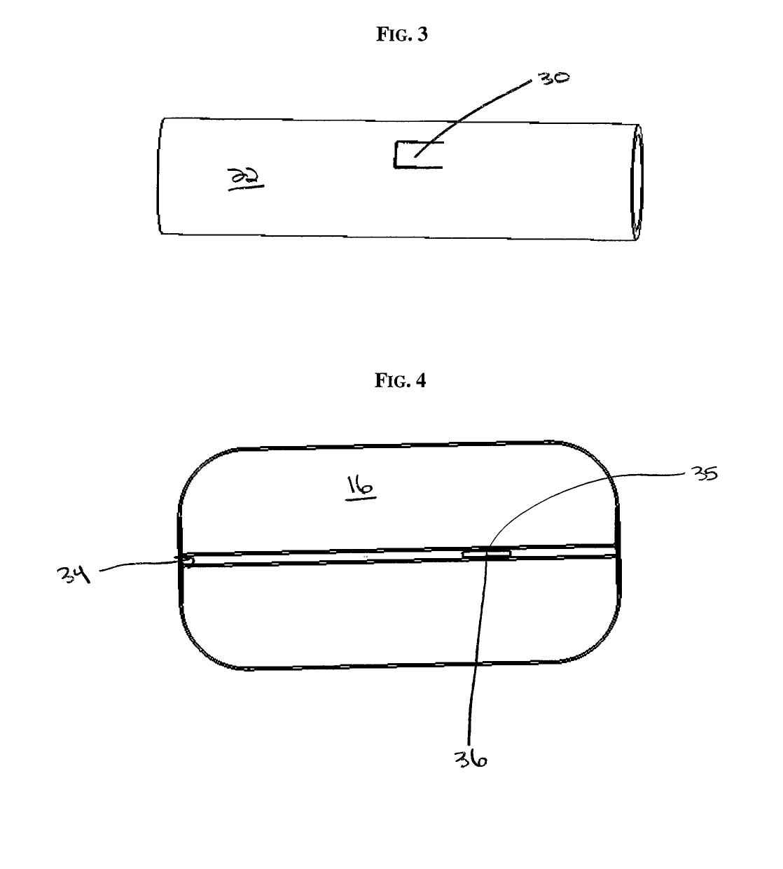

The expandable member 16 can also include features that can prevent the unintentional exit of fluid from the slit 36, resulting in the unintentional deflation of the expandable member 16. In one embodiment, a valve 22 can be positioned between the elongate member 12 and the wall 35 that forms the central opening 34, as discussed in more detail below. A variety of valves can be used, and in one embodiment, the valve is a one-way valve. FIG. 3 illustrates one embodiment of a one-way valve 22 having a flap 30 formed thereon. The valve 22 is adapted to be positioned such that the flap 30 surrounds the opening 24 and is adjacent to the slit 36. In use, fluid can flow from the lumen 20, through the opening 24, through the valve 22 and the slit 36, and into the expandable member 16. Pressure from the fluid in the expandable member 16 acts on the flap 30 to prevent back-flow through the valve 22 and out of the expandable member 16. While the exemplary embodiment illustrates a flap 30, one skilled in the art will appreciate the variety of other configurations that the one-way valve can have.

As noted above, at least a portion of the elongate member 12 is received within the central opening 34 to effect the delivery of fluid into the expandable member 16. The elongate member 12 can be secured within the central opening 34 using a variety of techniques, however in an exemplary embodiment an adhesive is used to enhance the connection. As shown in FIGS. 1C and 5, the distal end 12b of the elongate member 12 can be positioned within at least a portion of the central opening 34, such that the opening 24 in the lumen 20 is aligned with the slit 36 in the expandable member 16. The valve 22 can be positioned between the opening 24 and slit 36, such that the flap 30 opens up into the slit 36. In use, fluid can be delivered from the elongate member 12 to the expandable member 16 via the central opening 20. Fluid flows through the elongate member 12, out of the opening 24, and then through the valve 22 and the slit 36 to effect expansion of the expandable member 16.

As noted above, a mesh 18 can surround at least a portion of the expandable member 16. FIGS. 1A-1C illustrate a mesh 18 that surrounds the entire expandable member 16, such that the expandable member 16 is contained therein. In one embodiment, the mesh 18 has a pre-formed configuration such that it determines the shape of the expandable member 16 in the expanded position. While the configuration of the mesh 18 can vary depending upon the hollow organ that is to be occluded, when the hollow organ is the pyloric valve, the mesh 18 can have an enlarged portion that is adapted to be disposed within the stomach and a narrowed portion that is adapted to be disposed within the pyloric valve. In other embodiments, the configuration of the pre-formed mesh can be substantially cylindrical.

The mesh 18 can also be adapted to provide traction to the anchoring assembly 14 such that it can remain in a desired position in the hollow organ. For example, the mesh 18 can be formed in a pattern that is adapted to engage with tissue, such as interlocking circles, a cross-hatched pattern, a helical pattern, etc. One skilled in the art will appreciate that the mesh can be formed a variety of biocompatible materials, such as polyethylene and polyester.

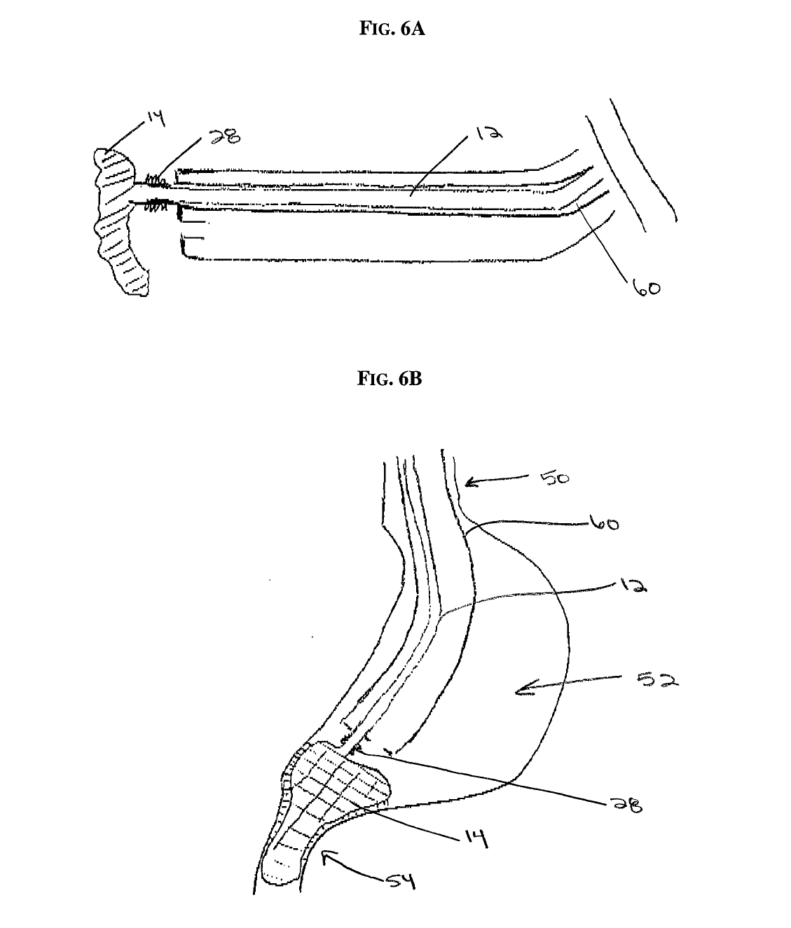

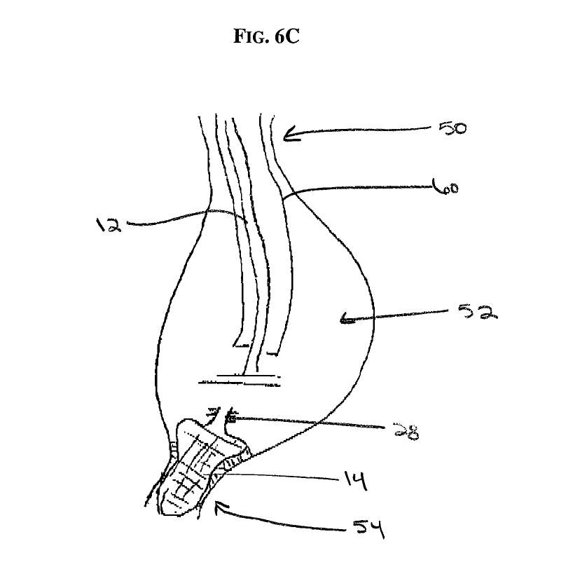

As previously indicated, the device 10 can be used to perform a variety of medical procedures. In an exemplary embodiment, however, the device 10 can be used to temporarily obstruct the pyloric valve prior to inflating the stomach of a patient during trans-gastric surgery. Following preparation of the patient as known in the art, the device 10 can be inserted into a natural or created orifice to a target site. As noted above, the device 10 is typically inserted in the delivery configuration shown in FIGS. 1A and 6A, where anchoring assembly 14 is in the deflated position. A variety of techniques can be used to insert the device 10 into the orifice, and in one embodiment, the device 10 can be positioned through an accessory channel of an insertion member 60, such as an endoscope. The insertion member 60 can be inserted down the esophagus 50 and into the stomach 52, and the device 10 can subsequently be delivered through the insertion member 60. Alternatively, the insertion member 60 and the device 10 can be delivered together to the stomach 52.

Once the distal end of the insertion member 60 is positioned within the stomach 52, a fluid can be delivered through the inner lumen 20 of the elongate member 12. A variety of biocompatible fluids can be used to inflate the expandable member 16, such as saline or air. The fluid then exits the opening 24 in the lumen 20, and passes through the slit 36 and the valve 22. This causes the expandable member 16 to expand. While the expandable member 16 can be expanded to a variety of diameters, in one embodiment, the expandable member 16 can be initially expanded to a diameter equal to or slightly less than that of the pyloric valve 54, e.g., in the range of about 1.0 cm to 2.0 cm. The expandable member 16 can subsequently be expanded again to a larger diameter such that it occludes the pyloric valve 54, as will be discussed in more detail below.

After the initial expansion of the expandable member 16, the insertion member 60 can be used to push the anchoring assembly 14 into the pyloric valve 54, or even a distance beyond it, to ensure complete obstruction, as shown in FIG. 6B. Once positioned within the pyloric valve 54, the expandable member 16 can be inflated to a larger diameter where it fills the mesh 18. As the expandable member 16 acts on the mesh 18, the mesh 18 expands to its pre-formed configuration. The pre-formed configuration of the mesh 18 will vary depending upon the type of hollow organ to be occluded, however when the organ is the pyloric valve 54, the mesh is preformed to a shape that has an enlarged portion adapted to be disposed within the stomach 52 and a narrowed portion adapted to extend into the pyloric valve 54. As a result, the portion of the anchoring assembly 14 disposed within the pyloric valve 54 can have an expanded diameter that is in the range of about 2.0 cm to 5.0 cm, while the portion of the anchoring assembly 14 that remains in the stomach 52 can have a diameter that is in the range of about 5.0 cm to 7.0 cm. Once inflated, the mesh 18 prevents the expandable member 16 from deforming as a result of pressure from the pyloric valve 54. As noted above, the mesh 18 also provides traction against the walls of the pyloric valve 54 to minimize movement of the anchoring assembly 14.

After the anchoring assembly 14 is operatively positioned and inflated, it can optionally be separated from the elongate member 12 at the frangible breaking point 28. Any endoscopic cutting device can be used to sever the elongate member 12 from the anchoring assembly 14, such as a grasper, etc. or a force can be applied to the elongate member 12 to pull the elongate member 12 apart from the anchoring assembly 14. As a result of separation, and as shown in FIG. 6C, the anchoring assembly 14 remains within the pyloric valve 54. The elongate member 12 can then be removed from the stomach 52 via one of the accessory channels of the insertion member 60. This frees space in the stomach 52, as well as the accessory channel, for the insertion and use of other instrumentation during the subsequent surgical procedure. While the exemplary method illustrates separation of the anchoring assembly 14 and the elongate member 12, in other embodiments, the device (e.g., the elongate member and the anchoring member) can remain intact throughout the entire surgical procedure.

After the surgical procedure is completed, the expandable member 16 can be deflated so that can be removed from the pyloric valve 54. In an exemplary embodiment, the expandable member 16 is deflated by puncturing it with any type of device that is pointed or able to perforate the expandable member 16. Once at least some of the fluid is drained from within the expandable member 16, the anchoring assembly 14 can be removed from the pyloric valve 54 and pulled back through an accessory channel of the insertion member 60 using, for example, any type of endoscopic grasper.

While the exemplary method illustrates a technique where the expandable member is inflated both during and after placement in the hollow organ, one skilled in the art will appreciate that in alternate methods the expandable member can be inflated only after placement in the hollow organ.

One skilled in the art will appreciate that the system and the device described herein can be processed before surgery. First, a new or used device and/or insertion member is obtained and if necessary cleaned. The device and/or the insertion member can then be sterilized. In one sterilization technique, the device can be placed in a closed and sealed container, such as a plastic or TYVEK bag. The container and the device are then placed in a field of radiation that can penetrate the container, such as gamma radiation, x-rays, or high-energy electrons. The radiation kills bacteria on the device and in the container. The sterilized device can then be stored in the sterile container, and the sealed container keeps the device sterile until it is opened in the medical facility. A similar technique can be used to sterilize the insertion member. In other embodiments, the device and/or the insertion member can be sterilized using any other technique that is known in the art, such as beta or gamma radiation, ethylene oxide, steam, etc.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical device, comprising:
an elongate member having a proximal end, a distal end, and an inner lumen extending through at least a portion thereof that is adapted to receive a fluid flow therethrough;
a selectively expandable member coupled to the distal end of the elongate member and adapted to receive fluid from the elongate member; and
a mesh disposed around at least a portion of the expandable member, the mesh having a pre-shaped configuration, at least a portion of which is adapted to be operatively positioned to occlude a portion of a hollow organ.

2. The device of claim 1, wherein the expandable member is annularly shaped, having a central opening in which the elongate member is disposed.

3. The device of claim 2, wherein the expandable member is elongate.

4. The device of claim 2, further comprising a slit that is formed within a wall of the expandable member that defines the central opening of the expandable member.

5. The device of claim 1, wherein a valve is disposed between an outlet of the inner lumen and an inlet of the expandable member.

6. The device of claim 5, wherein the valve is a one-way valve.

7. The device of claim 1, wherein the expandable member comprises a balloon.

8. The device of claim 1, wherein the expandable member is frangibly coupled to the distal end of the elongate member.

9. The device of claim 8, wherein elongate member includes a break point proximal to the expandable member that enables the expandable member to be frangibly coupled to the elongate member.

10. The device of claim 1, wherein the elongate member is flexible.

11. A kit for transgastric surgery, comprising:
an insertion member having at least one delivery channel extending therethrough, the insertion member being configured to be delivered translumenally to a surgical site within a patient;
an elongate member having proximal and distal ends and a fluid-conveying lumen extending through at least a portion thereof, the elongate member being configured to be removably disposed within the delivery channel; and
an anchoring member coupled to the distal end of the elongate member and including an expandable member contained within a mesh, at least a portion of the member having a configuration such that it is adapted to occlude a portion of a hollow organ when it is configured in an expanded position.

12. The kit of claim 11, wherein the mesh has an enlarged proximal portion and a narrowed distal portion.

13. The kit of claim 11, wherein the expandable member includes a one-way valve disposed between an outlet of the lumen and an inlet of the expandable member.

14. The kit of claim 11, wherein the anchoring member is frangibly coupled to the distal end of the elongate member.

15. The kit of claim 11, wherein the expandable member is fully contained within the mesh such that the expandable member conforms to a pre-shaped configuration of the mesh when the expandable member is expanded.

16. The kit of claim 11, wherein the insertion member comprises an endoscope.

17. A method for transgastric surgery, comprising:
positioning a portion of an anchor assembly located on a distal end of an elongate member within an opening of a pyloric valve of a patient's stomach; and
expanding an expandable member of the anchor assembly such that a mesh disposed around the expandable member engages and anchors the expandable member within the opening of the pyloric valve to temporarily block fluid flow from the stomach through the pyloric valve.

18. The method of claim 17, further comprising, prior to positioning, inserting the elongate member through an insertion member extending through a body lumen to position the anchor assembly within the stomach.

19. The method of claim 17, further comprising:
partially inflating the expandable member to a diameter that is substantially equal to a diameter of the pyloric valve;
pushing a portion of the expandable member into the pyloric valve; and
inflating the expandable member such that the mesh disposed around the expandable member engages and anchors the expandable member within the pyloric valve to block fluid flow from the stomach to the pyloric valve.
